(19) United States
(12) Patent Application Publication
(10) Pub. No.: US 2011/0077572 A1
(43) Pub. Date: Mar. 31, 2011

(54) Medical Equipment and Products using Chlorine, Hydrogen Peroxide, and Anti-Viral, Anti-Bacterial, Anti-Fungal, Agents, usage Method Improvements Dread Disease Cures, Remissions, and Anti-Infective Treatment Measures, Disease Treatment and Prevention System

(76) Inventor: Michael Robert Thomas, N. Ft. Myers, FL (US)

(21) Appl. No.: 12/625,740

(22) Filed: Nov. 25, 2009

Related U.S. Application Data
(60) Provisional application No. 61/117,625, filed on Nov. 25, 2008.

(30) Foreign Application Priority Data
Nov. 25, 2008 (US) 61117625

(51) Int. Cl. A61M 15/00; A61K 33/20; A01N 59/00; A61K 33/40; A61P 35/00; A61P 31/00; A01P 1/00; A61P 31/16; A01P 3/00; A61P 31/10; A61B 19/02; A61J 1/05; A61F 13/00; A61M 35/00

(52) U.S. Cl. 602/48; 424/661; 424/616; 128/203.29; 206/440; 604/403; 604/289

(57) ABSTRACT

Chlorine and Hydrogen Peroxide Medical Therapies including Ear Dropper Multiple Vile Medicine Mixing Cool Mist Vaporizer with detachable Inhalation Hose and Facemask Double Density Air Filter, 3-Speed Motor with On/Off Switch, Home and Hospital Models with Atomizer and an Orifice for attachment to and Oxygen Tank, Pre-Wrapped Bandages, Gauze Stick-On Strips or Squares or Strip-less Gauze Pads with Liquid or Gel, Chlorine or Hydrogen Peroxide also, Lotions for Skin Infections and Cancers, Chemical Injections and Patient Baths, and Wellness Therapies

### DRAWING DESCRIPTIONS AND SPECIFICATIONS NUMBERED

FIG. 1 a. Top View of a Cool Steam Vaporizer (1 to 5 gal. capacity) with Hose Piece and Mouth Piece for Direct Inhalation Therapy, Hospital and Home Model, with Capabilities of Adding Medicine to the Water Contained in the Vaporizer, b. Top View of Gauze Pads in Sterile Packs Sealed Foil, or if problems with hydrogen peroxide interaction with gauze or cotton, then sell in packages of 50% hydrogen peroxide and 50% gauze pads, Small Sizes 1″×1″ to 2″×2″, c. Gauze with Sterile Package Removed, Thick Model ¼″, Thin Model ⅛″, Treated with New or Existing Chemical Composition Gels or Liquid Chlorine or Hydrogen Peroxide in Various Strengths depending on usage and Clinical Study Results, d. Soaked or Gel Gauze Pad in Sterile Packs Sealed Foil, All Sizes including Larger Sizes 2″×2″ to 4″×4″, e. Soaked or Gel Gauze Pad with Sterile Package Removed, Thicknesses from ¼″ to ½″, Treated with New Chemical Compositions or Chlorine or Hydrogen Peroxide in Various Strengths depending on usage and Clinical Study Results, f. Side View of a cancer therapy or cyst removal or reduction machine that will work with potentially ultra-sound and multiple chemical agents using inter artery or artery insertion then vain retraction of chemicals to minimize negative or adverse effects on the patient, g. Front View of Chemical Synthesis Experiments, The potential usage of anti-viral agents such as various metal derivatives and chemical compounds, non-mercury type, and less toxic in nature chlorine alternatives, for the purpose of cancer therapies. Also, usable with this machine with be direct injection into the tumor core for the purpose of creating a drain hole after the injection occurs and the potential of multiple agent injections, h. hydrogen peroxide or chlorine pre-soaked gel or liquid soaked large size bandage, plastic or foil sealed, i. hydrogen peroxide or chlorine pre-soaked gel or liquid small size bandage, plastic or foil sealed, j. Top View of Bathtub narrow 3 ft. deep, hospital, commercial, whirlpool jet type with inside opening door for chlorine treatment anti-infective anti-cancer therapy hospital therapy treatments, k. chlorine dispensing unit adjustable dosage, I. Aloe, Vitamin E, and Orange Oil, with Hydrogen Peroxide or Chlorine (Controlled Low Dosage Therapy), or New and Existing Anti-Viral, Bacterial, or Fungal, Agents, for use as a lotion, m. Top View of Breathing Tube for Cool Steam Vaporizer without Attachments (This unit may be used with am elastic strapped face mask.) Lengths from 18″ to 8′, n. Top View of Breathing Tube for Cool Mist Vaporizer equipped with Squeeze Ball or Electric Atomizer/Nebulizer (This unit may be used with am elastic strapped face mask.) Lengths from 18″ to 8′, o. Top View of Atomizer/Nebulizer Squeeze Screw-On Cap Type or Electric Model for Inhalant Medicines, including but not limited to the individual usage or the usage in combination of the following prepared medical agents including; Aloe, Vitamin E, and Orange Oil, Chlorine and/or Hydrogen Peroxide (Low Dosage Therapy), New Liquid Hydro-gel Solutions and Anti-Biotic Solutions and Existing Anti-Viral and Anti-Bacterial Agents, with Special Low Dose Solutions of Chlorine and Hydrogen Peroxide, p. Orifice for attachment to an Oxygen Tank or Ventilator, q. Top View of Eye/Ear Dropper Bottles with Child Proof Caps for Custom Mixtures of Inhalation Therapies to be added to the Vaporizer as needed or directed by a Physician by removing the Vaporizer Motor, including but not limited to the individual usage or the usage in combination of the following prepared medical agents containing; Aloe, Vitamin E, and Orange Oil, Chlorine and/or Hydrogen Peroxide (Low Dosage Therapy), New Liquid Hydro-gel Solutions and Anti-Biotic Solutions and Existing Anti-Viral and Anti-Bacterial Agents, with Special Low Dose Solutions of Chlorine and Hydrogen Peroxide in Low Quantity Bottles under 6 oz. to assure Patient Safety with regards to the Danger of Overdosing using Standard Solutions, r. Removable Connector for Inhalation Tubes, Letters m. and n., when the adaptor is removed, Unit a. becomes a Standard Room Vaporizer, s. Side View of a Removable Fine Density Air Filter, t. Side View of a Removable Coarse Density Air Filter

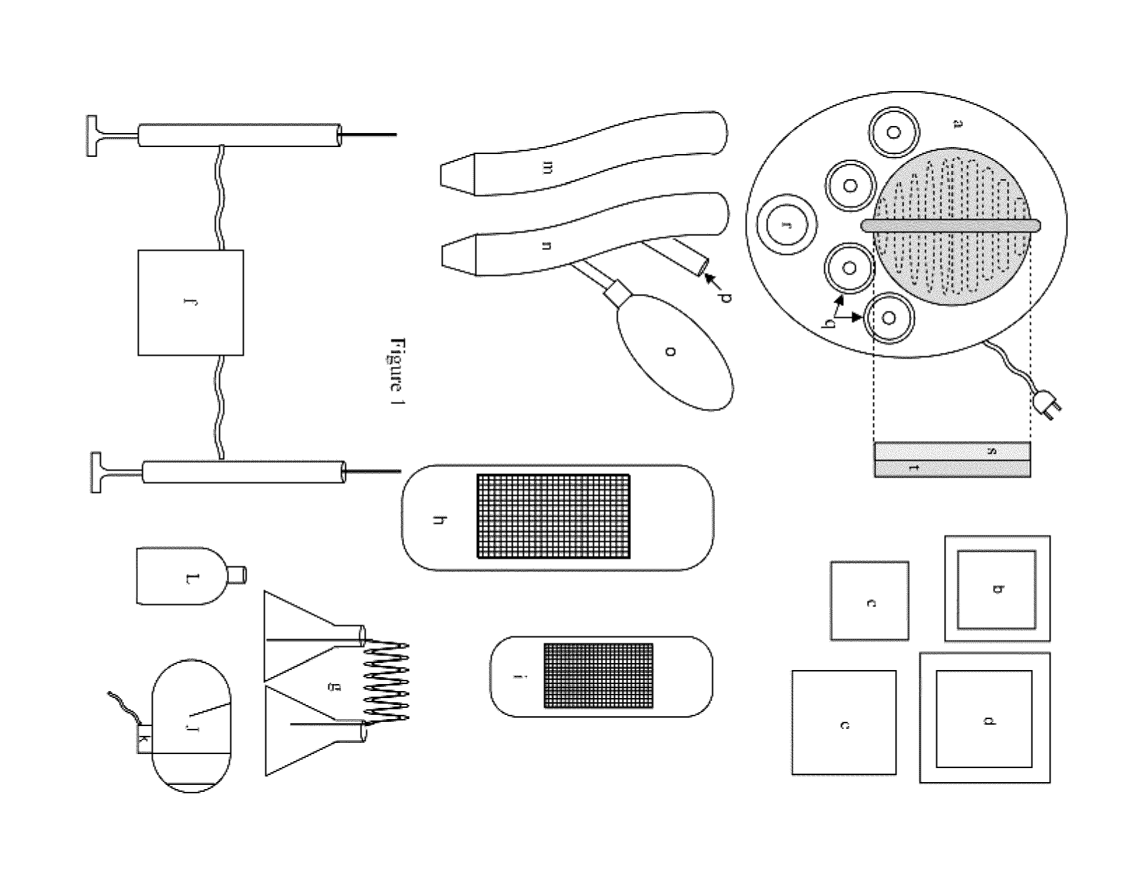

We reserve the right to retain as our own intellectual property any item that is determined not to be patentable in this or subsequent applications and the right to any compensation for the sale of that property and the right to receive compensation through any court system not connected with the USPTO.

### Anti-Conspiracy to Refuse to Deal Provision “At Odds Special Judicial Order Request”

We request that either now or in the future a special order be entered due to the fact that the manufacturing companies producing all of the products and services in the world are essentially at odds with this inventor due to patent conception disputes and therefore in order to continue human advancement it may be necessary to order financial partnership status, and that the patent be assigned to a company capable of producing it with an order of compensation attached in the amount of 15 to 50% after all expenses profits depending on what the judiciary feels is the correct compensation under the circumstances. This order is deemed necessary in order to stop companies from refusing to deal in order to conspire to obtain the patents for little or nothing due the fact that they have no need of additional monetary compensation profits from new products as they are extremely wealthy presently and this inventor has no financial backing and no previous art technical experience, although creating all of the major breakthroughs in all of the fields involved and receiving no compensation for them (Big Business Financial and Technical Monopolies). Companies refusing to abide by these terms should be closed down and their patentable items reassigned to inventor cooperative companies. Also, these new items should be considered separately from any previously disputed items.

Profit extraction methods as payment for the saving of lives and extending human life spans and the right to collect 15% of the profits from the following otherwise non-collectable medically beneficial concepts of all products and procedures contained in this patent and variations and products with omitted items developed by others after the priority date of this patent and its predecessor provisional version. Also included are metal derivatives other than mercury derivatives for the creation of less toxic anti-bacterial, anti-fungal, and anti-viral, agents, and the use of these or present anti-viral agents in conjunction with anything stated in this application or the right to 15% minimum of profits as compensation for the original creation of intellectual materials without which the items would not exist in so much that it is determined legal by the court systems either now or in the future. Also is the invention of physician wellness consulting concerning viral living organism human attack warnings by physicians to patients and the concept that viruses are attempting to kill animals and humans, and that humans need to stay in contact with chlorine or anti-viral agents in order to avoid and/or cure abnormalities and degenerations created by absence of the use of these agents (Chlorine or Die). Also the right to 15% of physician and hospital franchise charges associated with diagnoses and prescribing methods of cures and remissions contained is the patent using this medium used to produce patient wellness through awareness of these microscopic entities. Also is the conception of anti-infective treatments of tumors and infective conditions such as AIDS lesions and skin rashes, colon infection reduction, open spinal degeneration due to surgeries or injuries, other surgical infections, abnormalities, and diagnosed diseases using various strength chlorine baths, whirl pool type, and the concept of for profit medical billing associated with the coordinated treatments of chlorine and hydrogen peroxide therapies. Also the concept of hydrogen peroxide usage on infected areas before baths. Also the right to 15% or as adjudicated of profits associated with chorine manufacturing company profits after all expenses associated with increased demand for chlorine products and new purposes of usage associated with new uses and increased usage associated with this patent. Also the right for a re-issuance of patent concerning chlorine and hydrogen peroxide due to the large scale new medical improvements contained herein. Also is the right to 15% minimum of the profits for medical treatments using this technology in so much that it is determined legal by the court systems either now or in the future. Also the right to 15% minimum of the profits of other vaporizer manufacturers because patients may bypass the use of our invention and use the existing models that can be used in the same manner in so much that it is determined legal by the court systems either now or in the future. Also is the right to 15% minimum of the savings to health insurance companies with regards to savings to the companies themselves and to the patients that my theories throughout the years have produced in so much that it is determined legal by the court systems either now or in the future. Also is the right to 15% minimum of the profits after all expenses from the products contained herein due to the original creation of the product without which items would not exist, regardless of chemical composition, technical development, although if this arrangement is accepted, they may consider this a development partnership, in so much that it is determined legal by the court systems either now or in the future. Also the right to 15% minimum of any savings realized by the use of these inventions, in so much that it is determined legal by the court systems either now or in the future. Also the use of special packaging bottles for hospital and physician office standard and reduced dosages for medical usages patient contact products of hydrogen peroxide and chlorine solutions for the purpose of extracting a profit for the invention of the treatment concept and the right to charge higher prices for these substances in these medical usage packages.

### Disclaimer

We disclaim any responsibility or liability associated with any persons, companies, research groups, etc. who conduct unauthorized research or elect to treat themselves experimentally without our written permission, knowledge, consent, directions, approval, or without signed court recorded liability waivers. No persons are authorized to conduct and research without my court recorded and notified expressed written consent. A separate usage of hydrogen peroxide therapies and chlorine therapies as the inter mixing of the two agents has not been studied and may produce a toxic gas and/or neutralize the anti-infective capabilities of both agents or produce undocumented side effects. Clinical studies on animals will be required for all of these warnings and disclaimers.

### PURPOSE AND USEFULNESS

This invention will save lives and improve patient health and longevity by reducing or eliminating the procedure usage of breast mastectomy operations, lung removal operations, removal of infected internal organs, removal of cancerous internal organs tissue damaging chemo and radiation therapy, and associated pneumonia and influenza deaths.

The reduction of deaths and illness from bronchitis, asthma, emphysema, allergies, lung cancer, asbestosis, tuberculosis, influenza, colds, breast cancer, spinal degeneration, surgical infections, pneumonia, internal organ failure, ear and throat infections, nasal infection, eye and ear infections, surgical scar tissue breakdown deaths, jaw and gum infection, skin cancer, boils, etc.

The curing of all dread diseases will help to eliminate despair and depression regarding the loss of self and loved ones prematurely. Also more rewarding lives will be extended through increasing years of longevity, productivity, and healthiness. Also, suffering from viral attacks will be eliminated as a cause of death through increased awareness of viral attacks and changes in the medical field to an even more wellness related physician/patient relationship instead of the present theory of radical treatments that may be injurious or terminal to the patient. Clinical trials, studies, and the use of my previous invention of control groups will help to determine the accuracy of the theories contained in this application.

Also receiving benefit from the new invention will be medical insurance companies and those individuals with no insurance, and Medicare/Medicaid patients. Also, the inventions will reduce the amount of human suffering involved in chronic medical conditions associated with these viruses, funguses, and bacteria. Also reduced, will be long term suffering from the effects of radiation therapy, chemo therapy, and medical surgery.

What is claimed is:

1. Claimed is the invention of the 3-Speed with On/Off Switch Cool Mist Two Stage Density Washable Reusable Filtered Inhalation Therapy Vaporizer and Room Vaporizer Combination for household room model or hospital medical use model with detachable inhalation therapy hose or oxygen adding coupling and squeeze nebulizer model for antibiotic liquids or liquid stem cell therapies with detachable face mask, dual density filter, and recessed holes for medicine ear dropper plastic bottles for custom medicine mixing. Also, the use of stainless steel, glass, and low porosity plastic in the construction of commercial and hospital vaporizers for the purpose of reducing germ and virus growth potential porosity. Also the right for pharmacists and physicians to specifically prescribe this product, a hose type inhalant vaporizer, and restrict its sale to pharmaceutical prescription sales and usage, also the invention of a face mask and flexible hose usage with removable vaporizer adaptor in conjunction with a room vaporizer. Also, the invention of chemical compounds mixed with water and used in cool steam vaporizers directly inhaled to affect a cure or remission of lung diseases, where no previous cure or remission was associated with water vapor induced phlegm ejection through natural coughing over an extended period of time, such as common cold, influenza, pandemic influenza, pneumonia, tuberculosis, lung cancer, emphysema, asthma, bronchitis, and bacterial, fungus, dust, allergens, and industrial dust and fumes related diseases. Also, the discovery that cold air masses sweeping in from the north carry with them dust and particles the settle in the lungs within 12 to 24 hours of the high wind front, lows and highs causing the need for individuals to cleanse their lungs with water vapor from a cool mist water vaporizer in order eliminate these dust and viral particles thereby eliminating an influenza outbreak by purging and destroying the dust and viruses through the use of low dosage chlorine in the vaporizer water and the fact that the additional moisture in the lungs creates phlegm that is subsequently coughed up after a period of hours or days, usually a period of several hours to several days, also the invention of a treatment regiment in order to safely use the chlorine in conjunction with the vaporizer through warning labels on the vaporizer, the box and the instructions. Also claimed is the concept increasing dosage instructions connected with the vaporizer that tell the patient to gradually increase both the concentration of the chlorine and the length of water vapor inhalation in order to not create a too heavy of a concentration of either chlorine or water vapor in the lungs.

2. Claimed is the invention of the usage of high and low concentrations of chlorine and hydrogen peroxide and antibiotic ointments used in combination and/or used individually and other known or unknown anti-viral or cleansing chemical agents for the purpose of shrinking cancer tumors into a state of remission or cure, and the concept of repeat dosages in order to maintain the remission, cure, or prolonged life span, through prevention of bacterial, fungal, or viral, tissue infection degeneration associated with inability of the infection to thrive in the patient infection body weakness areas caused by continuing treatments (1 capful in a whirlpool bath as in infection beneficial dose). Also the use of wash cloth and water bowl for administering shock dosages to particular body areas of chlorine or hydrogen peroxide. These treatments may also be experimented for tuberculosis, influenza, pandemic influenza, cancers, such as, lung cancer, throat cancer, stomach and intestinal cancers, and colon cancer, skin cancers, viral, bacterial, fungal, leprosy, tetanus, rabies, AIDS, and other infections in appropriate doses for the situation, Also, the invention of the theory of initial heavy shock doses for infection control and light long term doses of chlorine for infection control and curing or reducing cancer tumors, cysts, and healing rate improvements. The theory involves a belief that nearly instantaneous shrinking will begin once the chlorine is applied and over a period of weeks to months, the tumor will shrink to become in remission thereby eliminating the need for invasive surgical procedures to be performed that create delayed deaths of the patient due to the inability to survive or thrive without the affected tissues remaining in the body. Likewise the use of radiation may weaken the cells to the point of tissue breakdown and subsequently premature deaths due to the long term breakdown of the tissues. Also the usages of hydrogen peroxide and chlorine as co-agents whether mixed or applied in separate applications along with chlorine in a regiment of usage over a period of weeks, months, or years, as the cancers cells attempt to reverse the remission. Also the use of chlorine and hydrogen peroxide during and after surgery to reduce infection rates and treat infections that exist in patients after hospital admissions, also increased life span years, possibly seven or longer, after the necessity of severe surgery or injury through daily treatment in years following, also the concept of washing off heavy concentrations of chlorine to encourage quicker healing after initial applications, also the invention of chlorine and or hydrogen peroxide for the purpose of cyst or cancerous tumor remission and cure as opposed to surgical removal. The new vaporizer usage has the potential after testing to kill influenza directly in the lungs through the usage of low dosage chlorine inhalation, Also the use of chlorinated medical whirlpool spa for curing and controlling otherwise uncontrollable infection, internally and externally. Also the medical usage sales of chlorine for stocking in hospitals, physician offices, pharmacies, and veterinarian facilities, including percentage dilutions such as; 2%, 5%, 10%, 25%, and 50%, of full strength chloride and peroxide standard dilutions for direct application medical usage and the right to restrict sales to these medical facilities.

3. Claimed is the invention of cure, remission, or non-progression, of degenerative conditions through the use of antibiotic ointments and gels, and antiviral, bacterial, anti-fungal, and chemical agents. This concept identifies the potential that degenerative conditions maybe be aided by external deep healing hydrogen peroxide therapy for heart damage or ligament and cartilage joint injuries and the use or continued used of low dosage chlorine baths and high dosage injections, low to high dosage lotions or medicated pads and inhalation therapies of low dosage chlorine and or the treatment of breast tumors, throat tumors, joint tumors, spinal degeneration, lung tumors, and other cyst or cancer, through injection of the tumor. Also, the concept of artery injections in localized areas in order to affected a treatment of hydrogen peroxide or chlorine or a combination of anti-viral chemical agents. Also after testing, pandemic influenza is likely curable through the usage of vaporizer low dosage chlorine inhalation therapy using dosages in the area of two to eight drops per gallon three times daily for 30 seconds to 3 minutes, discontinue or reduce treatment frequency to determine optimal frequency by discontinuing treatment for 1 to 5 days if patient experiences unmanageable phlegm volume. The patient should start with the lowest chlorine concentration and start with the lowest inhalation time. In order to determine the threshold level between beneficial treatment and lung injury from the toxic affects of the chemical agent. Hydrogen peroxide experimental low dosage therapy could also be used in a similar manner in instances where chemical, smoke, or dust, deposits may require a foaming agent to help create phlegm so the patient can cough up the detrimental substances.

4. Claimed is the invention of the development of chlorine, hydrogen peroxide, aloe oil, and orange oil, as ingredients for skin lotions and sun tan lotions and oils as a treatment for skin cancers due to the killing of viruses, germs, and funguses through skin contact with chlorine and hydrogen peroxide. Also the invention of lotions containing chlorine and/or hydrogen peroxide and the potential cure or reduction of skin cancer symptoms associated with the usage of these lotions, also the use of aloe oil and orange oil in lotions for the purpose of skin moisturizers, also the invention of aloe oil, orange oil, chlorine, and hydrogen peroxide, for the purpose of inhalation therapies, also, the invention of antibiotic liquids for use in vaporizers.

5. Claimed is the invention of medical products including chlorine and hydrogen peroxide bandages soaked, also chlorine and hydrogen peroxide gel, also combination of hydrogen peroxide and existing formula antibiotic petroleum ointment, all products individually sealed with plastic or foil, also packaged plastic stick tape backed square type and rectangle strip stick-on type bandages and un-backed gauze pads. Also the invention of tubes of existing formula antibiotic gel ointment, also tubes of hydrogen peroxide petroleum ointment, also tubes of hydrogen peroxide gel, also tubes of chlorine gel, also tubes of chlorine petroleum ointment. Also claimed are the inventions of hydrogen peroxide gel and the invention of chlorine gels for medical usage in conjunction with existing dry bandages and gauze pads. Also claimed is the invention of the antiseptic usage of these new types of products.
